(19) United States
(12) Patent Application Publication
(10) Pub. No.: US 2012/0171181 A1
(43) Pub. Date: Jul. 5, 2012

(54) COMPOSITIONS AND MINIMALLY INVASIVE METHODS FOR TREATING CANCER

(76) Inventor: Allan Mishra, Menlo Park, CA (US)

(21) Appl. No.: 13/393,012

(22) PCT Filed: Aug. 31, 2010
(86) PCT No.: PCT/US10/47393

§ 371 Date: Feb. 28, 2012

Related U.S. Application Data
(60) Provisional application No. 61/239,892, filed on Sep. 4, 2009.

(51) Int. Cl. A61K 35/16; A61P 35/00; A61K 35/12

(52) U.S. Cl. 424/93.72; 424/93.7

(57) ABSTRACT

Methods are described for using compositions containing platelet-rich plasma for the treatment of a cancer in a variety of tissues. Particularly, treatment of brain cancer with platelet releasate is described.

### RELATED APPLICATIONS

This application claims priority to U.S. Provisional Application No. 61/239,892, filed Sep. 4, 2009, which is incorporated herein by reference.

### BACKGROUND OF THE INVENTION

1. Field of the Invention

Embodiments of the present invention are directed to compositions and methods for the treatment of a cancer in any type of tissues. In a preferred embodiment, the cancer subject to claimed compositions and treatments is brain cancer.

2. Description of the Related Art

The term “cancer” or “tumor” encompasses a spectrum of diseases that vary in treatment, prognosis, and curability. The approach to diagnosis and treatment depends on the site of tumor origin, the extent of spread, sites of involvement, the physiologic state of the patient, and prognosis. Once diagnosed, the tumor is usually “staged,” a process which involves using the techniques of surgery, physical examination, histopathology, imaging, and laboratory evaluation to define the extent of disease and to divide the cancer patient population into groups in order of decreasing probability of cure. Such systems are used both to plan treatment and determine the prognoses for the patient (Stockdale, F., 1996, “Principles of Cancer Patient Management,” In: Scientific American Medicine, vol. 3, Dale, D. c., and Federman, D. D. (cds.), Scientific American Press, New York).

The type or stage of the cancer can determine which of the three general types of treatment will be used: surgery, radiation therapy, and chemotherapy. An aggressive, combined modality treatment plan can also be chosen. To this end, surgery can be used to remove the primary tumor, and the remaining cells are treated with radiation therapy or chemotherapy (Rosenberg, S. A., 1985, “Combined-modality therapy of cancer: what is it and when does it work?” New Engl. J. Med. 312:1512-14).

Cancer can occur in almost all types of tissues. Some examples of such tissues include connective tissues, prostate, lung, colon, bladder, skin, pancreas, blood cells, breast, ovary, gonad and brain.

In particular, a brain cancer can be caused by abnormal and uncontrolled cell division in brain itself and cells/tissues nearby brain (primary brain cancer) or it can be caused via metastases of tumors from other organs (secondary, metastatic brain cancer). The primary brain cancer may occur in neurons, glial cells, lymphatic tissue, blood vessels, cranial nerves, brain envelopes, skull, pituitary and pineal glands. Primary brain cancers are commonly located in the posterior cranial fossa in children and in the anterior two-thirds of the cerebral hemispheres in adults, although they can affect any part of the brain.

Brain cancer is the leading cause of cancer-related death in patients younger than age 35. Primary brain cancers account for 50% of intracranial tumors and secondary brain cancer accounts for the remaining cases. Approximately 17,000 people in the United States are diagnosed with primary cancer each year and nearly 13,000 die of the disease. Secondary brain cancer occurs in 20-30% of patients with metastatic disease and incidence increases with age. In the United States, about 100,000 cases of secondary brain cancer are diagnosed each year.

Surgery plays the central role in the diagnosis and treatment of cancer. In general, a surgical approach is required for biopsy, and surgery can be the definitive treatment for most patients with cancer. Surgery is also used to reduce tumor mass, to resect metastases, to resolve medical emergencies, to palliate and rehabilitate. Although the primary surgical technique for cancer field where tumors are resected under direct visualization, current techniques allow for some resections to be performed by endoscopic means. A primary concern in the treatment of cancer is the consideration of operative risk (Stockdale, F., supra).

Radiation therapy plays an important role in both the primary and palliative treatment of cancer. Both teletherapy (megavoltage radiation therapy) and brachytherapy (interstitial and intracavity radiation) are in common use. Electromagnetic radiation in the form of x-rays is most commonly used in teletherapy to treat common malignant tumors, while gamma rays, a form of electromagnetic radiation similar to x-rays but emitted by radioactive isotopes of radium, cobalt, and other elements, are also used. Radiation therapy transfers energy to tissues as discrete packets of energy, called photons, that damage both malignant and normal tissues by producing ionization within cells. The target for the ions is most commonly the DNA; radiation therapy exploits the fact that the radiation damage is not uniform between malignant and non-malignant tissues and rapidly dividing cells are more sensitive to DNA damage than quiescent cells (Pass, H. 1., 1993, “Photodynamic therapy in oncology: mechanisms and clinical use,” J. Natl. Cancer Instit. 85:443-56.) Radiation therapy is associated with unique benefits as well as important toxicities. Radiation is preferred in certain anatomic areas, (e.g., the mediastinum), where radiation may be the only feasible local method of treatment, and radiation may also be the only feasible local modality if tumor involvement is extensive. Radiation may also be used when the patient finds surgery unacceptable, or when the patient's medical condition prohibits a surgical procedure. Radiation treatment involves tissue damage which can lead to early and late radiation effects. The early effects (acute toxicity of radiation therapy) include erythema of the skin, desquamation, esophagitis, nausea, alopecia, and mylosupression, while the late effects include tissue necrosis and fibrosis, and usually determine the limiting toxicity of radiation therapy (Stockdale, F., supra).

Nearly all chemotherapeutic agents currently in use interfere with DNA synthesis, with the provision of precursors for DNA and RNA synthesis, or with mitosis, and thus target proliferating cells (Stockdale, F., “Cancer growth and chemotherapy,” supra). Animal tumor investigation and human clinical trials have shown that drug combinations produce higher rates of objective response and longer survival than single agents (Frei, E. III, 1972, “Combination cancer therapy: presidential address,” Cancer Res. 32:2593-2607). Combination drug therapy uses the different mechanisms of action and cytotoxic potentials of multiple drugs, including the alkylating agents, antimetabolites, and antibiotics (Devita, V. T., et al., 1975, “Combination versus single agent chemotherapy: a review of the basis for selection of drug treatment of cancer,” Cancer 35:98-110). The physiologic condition of the patient, the growth characteristics of the tumor, the heterogeneity of the tumor cell population, and the multidrug resistance status of the tumor influence the efficacy of chemotherapy. Generally, chemotherapy is not targeted (although these techniques are being developed, e.g. Pastan, 1. et al., 1986, “Immunotoxins,” Cell 47:641-648), and side effects such as bone marrow depression, gastroenteritis, nausea, alopecia, liver or lung damage, or sterility can result.

The treatment regimes described above have had varying degrees of success. Because the success rate is far from perfect in many cases research continues to develop better treatments.

One promising area of research relates to affecting the immune system. By the use of genetic engineering and/or chemical stimulation it is possible to modify and/or stimulate immune responses so that the body's own immune system treats the disease e.g., antibodies destroy cancer cells. This type of treatment departs from those described above in that it utilizes a biological process to fight a disease. However, the treatment is still a treatment that involves giving the patient an active compound not native to the patient.

The methods and compositions described herein are directed to a new approach in cancer treatment which can be used alone or in connection with the technologies described above.

The treatments described herein relate to treatment of cancerous tissues such as a tumor with platelet rich plasma. In some cases, the platelet-rich plasma may be prepared from the patient's own blood or from a donor. By the methods described herein, the blood and/or components from a patient or donor can be processed and used to treat cancerous cells.

### SUMMARY OF THE INVENTION

In one embodiment, the present invention is drawn to a method of treating a cancer in an individual including the steps of determining a site of the cancer in the individual; and introducing a platelet-rich plasma composition into and around the site of the cancer. In preferred embodiments, the tissue is selected from the group consisting of connective tissues, prostate, lung, colon, bladder, skin, pancreas, blood cells, breast, ovary, gonad, brain or any combination thereof. In a preferred embodiment, the tissue is a brain tissue.

In some embodiments, the platelet-rich plasma composition is treated with energy waves such as ultrasound to release platelet contents.

In preferred embodiments the platelet-rich plasma is titrated to obtain a pH of about 7.3 to 7.5. In a more preferred embodiment, the titration is performed using a bicarbonate buffer.

In preferred embodiments, the platelet-rich plasma composition includes platelets obtained from the individual who is to be treated with the platelet-rich plasma composition. In some embodiments, no exogenous activator is added to the composition prior to its introduction into and around the site of cancer.

In some embodiments, the method includes the step of mixing into the platelet composition substantially simultaneously with the introduction into and around the site of cancer, one or more ingredients including, but not limited to, thrombin, epinephrine, collagen, calcium salts, and pH adjusting agents. In some embodiments, the platelet-rich plasma composition is depleted in neutrophils.

In one embodiment the present invention is drawn to a platelet-rich plasma composition for the treatment of cancer which includes platelet-rich plasma; and a pH adjusting agent, wherein the composition may or may not contain an activator of the platelet-rich plasma. In a preferred embodiment, the pH of the platelet-rich plasma composition is adjusted to a pH of about 7.3 to 7.5 with a pH adjusting agent. In a more preferred embodiment, the pH adjusting agent is a bicarbonate buffer. Preferably, the plasma used for the platelet-rich plasma is from an autologous source.

In one embodiment, the present invention is drawn to a method of making a platelet-rich plasma composition including the steps of drawing blood from an individual; obtaining a plasma fraction from the blood; isolating platelets from the plasma fraction; resuspending the platelets in a reduced amount of plasma; and adjusting the pH to provide a pH of 7.3 to 7.5 for the resuspended platelets to provide a platelet-rich plasma composition, wherein an activator of the platelet-rich plasma may or may not be added to the platelet-rich plasma composition.

Embodiments of the invention are directed to methods for treating a cancer by obtaining cancer cells from an individual, culturing the cancer cells in a medium that includes a platelet extract; and introducing at least some of the cancer cells cultured in the medium with the platelet extract to an area of cancerous tissue in an individual thereby treating cancer. Preferably, the platelet extract is an autologous platelet extract obtained from the individual. Preferably, the platelet extract is buffered to a physiological pH, more preferably, the physiological pH is between about 7.3 and 7.5. In preferred embodiments, a buffer such as sodium bicarbonate, calcium gluconate, choline chloride, dextrose, EGTA, HEPES, maleic acid, MOPS, PIPES, sucrose, TES, TRIS BASE, TRIS-HCl, or urea is used to buffer the platelet-rich plasma composition.

In some embodiments, the composition also includes an agent such as growth factors, growth factor inhibitors, NSAIDS, steroids, anti-infectives, or some combination of the above In some preferred embodiments, the platelet rich plasma in the composition is depleted in neutrophils.

Preferably, introducing the cancer cells is by catheter, needle or any functional equivalent.

Preferred embodiments are directed to methods for treating a cancer by obtaining non-cancerous cells from a first individual, culturing the non-cancerous cells in a medium with platelet extract, and introducing at least some of the non-cancerous cells cultured in the medium with platelet extract to an area of cancerous tissue in the first or a second individual thereby treating cancer.

Preferably, the platelet extract is an autologous platelet extract obtained from the individual. Preferably, the platelet extract is buffered to a physiological pH, more preferably, the physiological pH is between about 7.3 and 7.5. In preferred embodiments, a buffer such as sodium bicarbonate, calcium gluconate, choline chloride, dextrose, EGTA, HEPES, maleic acid, MOPS, PIPES, sucrose, TES, TRIS BASE, TRIS-HCl, or urea is used to buffer the platelet-rich plasma composition.

In some embodiments, the composition also includes an agent such as growth factors, growth factor inhibitors, NSAIDS, steroids, anti-infectives, or some combination thereof. In some preferred embodiments, the platelet rich plasma in the composition is depleted in neutrophils.

Preferably, introducing the cancer cells is by catheter, needle or any functional equivalent.

Embodiments are directed to the use of a composition which includes platelet-rich plasma for the preparation of a medicament for treating cancer. In some embodiments, the platelet rich plasma is treated with energy waves prior to delivery to an area of cancerous tissue.

In some embodiments, the platelet rich plasma composition is adjusted to physiological pH, such as pH 7.3 to 7.5. The pH may be adjusted with a buffer such as sodium bicarbonate, calcium gluconate, choline chloride, dextrose, EGTA, HEPES, maleic acid, MOPS, PIPES, sucrose, TES, TRIS BASE, TRIS-HCl, or urea.

In some embodiments, the composition also includes an agent such as growth factors, growth factor inhibitors, NSAIDS, steroids, anti-infectives, or some combination thereof.

In some embodiments, the platelet rich plasma in the composition is depleted in neutrophils.

### BRIEF DESCRIPTION OF THE DRAWINGS

These and other feature of this invention will now be described with reference to the drawings of preferred embodiments which are intended to illustrate and not to limit the invention.

FIG. 1 shows human glioblastoma cells grown in cell culture media for 10 days. Magnification is 200×.

FIG. 2 shows human glioblastoma cells grown in cell culture media with 10% platelet releasate for 10 days.

FIG. 3 shows human glioblastoma cells grown in cell culture media with 10% platelet releasate for 10 days. The arrow indicates an astrocyte cell.

### DETAILED DESCRIPTION OF THE PREFERRED EMBODIMENT

Some embodiments of the present invention provide compositions and methods for treating cancers, in particular, brain cancer. Such compositions and methods can be used alone or in combination with treatments available in the art such as surgery, radiation therapy, chemotherapy, immunological treatments or any combination thereof.

Before the present formulations and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a cell” includes a plurality of such cells and reference to “the method” includes reference to one or more methods and equivalents thereof known to those skilled in the art, and so forth.

The term “platelet” is used here to refer to a blood platelet. A platelet can be described as a minuscule protoplasmic disk occurring in vertebrate blood. Platelets play a role in blood clotting. The platelet may be derived from any source including a human blood supply, or the patient's own blood. Thus, the platelets in the composition of the inventions may be autologous. The platelets may be homologous, i.e. from a human but not the same human being treated with the composition.

The term “platelet-rich-plasma”, “PRP” and the like are used interchangeable here to mean a concentration of platelets in a carrier which concentration is above that of platelets normally found in blood. For example, the platelet concentration may be 5 times, 10 times, 100 times or more the normal concentration in blood. The PRP may use the patient's own plasma as the carrier and the platelets may be present in the plasma at a range of from about 200,000 or less to 2,000,000 or more platelets per cubic centimeter. The PRP may be formed from whole blood e.g. by technology disclosed in any of U.S. Pat. Nos. 5,614,106; 5,580,465; 5,258,126 or publication cited in these patents and if needed stored by technology as taught in U.S. Pat. No. 20021 0034722A1; 5,622,867 or publications cited therein. The PRP may comprise blood component other than platelets. It may be 50% or more, 75% or more, 80% or more, 95% or more, 99% or more platelets. The non-platelet components may be plasma, white blood cells and/or any blood component. PRP is formed from the concentration of platelets from whole blood, and may be obtained using autologous, allogenic, or pooled sources of platelets and/or plasma. PRP may be formed from a variety of animal sources, including human sources.

The “dose” of platelets administered to a patient will vary over a wide range based on the age, weight, sex and condition of the patient as well as the patients' own normal platelet concentration, which as indicated above can vary over a ten fold or greater range. Doses of 1 million to 5 million platelets are typical but may be less or greater than such by a factor of two, five, ten or more.

The term “platelet releasate” is the PRP as defined above but treated so that what is inside the platelet shells is allowed to come out. The releasate may be subjected to processing whereby the platelet shells are removed and/or other blood components are removed, e.g. white blood cells and/or red blood cells or remaining plasma is removed. For example, the platelets may be treated with ultrasound to open the platelets and release the contents. The pH of the platelet releasate may be adjusted to physiological pH or higher or to about 7.4±10%, 7.4±5%, 7.4±2% or 7.4 to 7.6 as needed.

The terms “treatment”, “treating” and the like are used herein to generally mean obtaining a desired pharmacologic, physiologic or cosmetic effect. The effect may be prophylactic in terms of completely or partially preventing a condition, appearance, disease or symptom thereof and/or may be therapeutic in terms of a partial or complete cure for a condition and/or adverse effect attributable to a condition or disease. “Treatment” as used herein covers any treatment of a condition, disease or undesirable appearance in a mammal, particularly a human, and includes:
(a) inhibiting the disease, condition or appearance, i.e., causing regression of condition or appearance; and/or
(b) relieving the disease, condition or undesired appearance, i.e., causing regression of condition or appearance.

The invention includes treating patients with platelet releasate or components thereof formulated in accordance with the invention. Accordingly, the term “treatment” is intended to mean providing a therapeutically detectable and beneficial effect of any kind on a patient.

The inventive platelet composition may be a biocompatible composition that comprises unactivated platelets, activated platelets, platelet releasate(s), or the like. In an embodiment, the inventive platelet composition comprises platelet-rich plasma (PRP). In some embodiments, the PRP composition is depleted in neutrophils. The PRP may be activated or unactivated. Optionally, the PRP composition is treated with ultrasound to release contents.

Platelets are cytoplasmic portions of marrow megakaryocytes. They have no nucleus for replication; the expected lifetime of a platelet is some five to nine days. Platelets are involved in the hemostatic process and release several initiators of the coagulation cascade. Platelets also release cytokines and growth factors involved with initiating wound healing.

A wide variety of cytokines and growth factors are released by platelets. Some non-limiting examples of such cytokines and growth factors are platelet derived growth factor (PDGF), transforming growth factor-beta (TGF-b), platelet-derived angiogenesis factor (PDAF), platelet derived endothelial cell growth factor (PD-ECGF), insulin-like growth factor (IGF) and others. These cytokines and growth factors serve a number of different functions including helping to stimulate cell division at an injury site. The growth of cells is affected by the molecules such as growth factors and cytokines. Cells respond to particular types of growth factors or cytokines and have their growth and proliferation modulated. The combination of molecules most suitable for modulating cellular growth and proliferation are found in the animal's own platelets as well as other isolated components of blood. Growth factors and cytokines also work as powerful chemotactic factors for mesenchymal cells, monocytes and fibroblasts, among others.

Historically, PRP has been used to form a fibrin tissue adhesive through activation of the PRP using thrombin and calcium, as disclosed in U.S. Pat. No. 5,165,938 to Knighton, and U.S. Pat. No. 5,599,558 to Gordinier et al., incorporated in their entirety by reference herein. Activation results in release of the various cytokines and also creates a clotting reaction within various constituents of the plasma fraction. The clotting reaction rapidly forms a platelet gel (PG) which can be applied to various wound surfaces for purposes of hemostasis, sealing, and adhesion.

The platelet composition may be prepared using any conventional method of isolating platelets from whole blood or platelet-containing blood fractions. These include centrifugal methods, filtration, affinity columns, and the like. If the platelet composition comprises PRP, then conventional methods of obtaining PRP, such as those disclosed in U.S. Pat. Nos. 5,585,007 and 5,788,662 both to Antanavich et al., incorporated herein by reference in their entirety, may be utilized.

The formulation of PRP may vary according to the needs of the specific cancer to be treated. The PRP, for example, could be activated with thrombin and or calcium prior to use or could be used in an unactivated form. Ultrasound may or may not be used to create a platelet releasate that may then in turn be used to treat cancer.

In preferred embodiments, the PRP composition is depleted in granulocytes. In particular, neutrophils are depleted, preferably at a level of 0 to 0.9 of the concentration of whole blood. In some variations, the neutrophil concentration may be between about 0.01 and about 0.1 times baseline, about 0.1 and about 0.5 times baseline, or about 0.5 and 1.0 times baseline, where baseline is understood to be the level of neutrophils in whole blood. The neutrophil concentration may additionally or alternatively be specified relative to the concentration of the lymphocytes and/or the monocytes. One microliter of whole blood typically comprises 2,000 to 7,500 neutrophils. In some variations, the PRP composition may comprise neutrophils at a concentration of less than about 1,000 per microliter, about 1,000 to about 5,000 per microliter, or about 5,000 to about 7,500 per microliter. In preferred embodiments, neutrophils are eliminated or substantially eliminated. Means to deplete blood products, such as PRP, of neutrophils is known as discussed in U.S. Pat. No. 7,462,268, which is incorporated herein by reference.

The PRP, or a portion of the PRP, may be placed into an automated blood analyzer that performs a compete blood count (CBC). As part of the CBC, the automated blood analyzers typically return a count of the number of platelets, WBCs, and RBCs present in the sample. The WBC count may further include counts of lymphocytes, monocytes, basophils, neutrophils, and/or eosinophils. Examples of blood analyzers that may be used include, but are not limited to, Beckman Coulter LH series, Sysmex XE-2100, Siemens ADVIA 120 & 2120, and the Abbott Cell-Dyn series. The PRP may then be modified to contain specific ratios of platelets to other blood components such as neutrophils.

The PRP can be prepared using a variety of techniques including, but not limited to, centrifuges, gravity filtration devices, cell sorting or others. The PRP can be made and then stored in a frozen or lyophilized state. In a preferred form it would be buffered to physiologic pH. The PRP could be prepared in a form that is depleted of neutrophils, either partially or completely.

The platelet-rich plasma composition may be delivered to an individual in need thereof by convention as a means which include injection using a syringe or catheter, The platelet rich plasma composition may also be delivered via a dermal patch, a spray device or in combination with an ointment, bone graft or drug. It may further be used as a coating on suture, stents, screws, plates or some other implantable medical device. Finally, it may be used in conjunction with a bioresorbable drug or device.

The site of delivery of the platelet-rich plasma composition is at or near the site of cancer cell growth. The site of cancer is determined by well-established methods including imaging studies. The preferred imaging study used is determined by the tissue type. Commonly used imaging methods include, but are not limited to, MRI, X-ray, CT scan, Positron Emission tomography (PET), Single Photon Emission Computed Tomography (SPECT), Electrical Impedance Tomography (EIT), Electrical Source Imaging (ESI), Magnetic Source Imaging (MSI), laser optical imaging and ultrasound techniques.

Adjusting the pH of platelet compositions has been used to prolong the storage time of unactivated platelets, as disclosed in U.S. Pat. No. 5,147,776 to Koerner, Jr. and U.S. Pat. No. 5,474,891 to Murphy, incorporated by reference herein. pH may be adjusted using a variety of pH adjusting agents, which are preferably physiologically tolerated buffers, but may also include other agents that modify pH including agents that modify lactic acid production by stored platelets. Especially useful are those pH adjusting agents that result in the pH of the platelet composition becoming greater than or equal to physiological pH. In an embodiment, the pH adjustment agent comprises sodium bicarbonate. Physiological pH, for the purposes of this invention, may be defined as being a pH ranging from about 7.35 to about 7.45. pH adjusting agents useful in the practice of this invention include bicarbonate buffers (such as sodium bicarbonate), calcium gluconate, choline chloride, dextrose (d-glucose), ethylenebis(oxyethylenenitrilo)tetraacetic acid (EGTA), 4-(2-hydroxyethyl)-1-piperazineethanesulfonic acid (HEPES), maleic acid, 4-morpholinepropanesulfonic acid (MOPS), 1,4-piperazinebis(ethanesulfonic acid) (PIPES), sucrose, N-tris(hydroxymethyl)methyl-2-aminoethanesulfonic acid (TES), tris(hydroxymethyl)aminomethane (TRIS BASE), tris(hydroxymethyl)aminomethane hydrochloride (TRIS.HCl), and urea. In a preferable embodiment, the pH adjusting agent is a bicarbonate buffer, more preferably, sodium bicarbonate.

In one aspect, the invention relates to a method of treating a cancer comprising: obtaining a platelet composition; determining location of cancer; and minimally invasively introducing the platelet composition into and around the cancer. In an aspect, the invention relates to the method wherein the platelet composition is at or above physiological pH. In an aspect, the invention relates to the method wherein the platelet composition optionally includes platelet releasate. In an aspect, the invention relates to the method further comprising: mixing into the platelet composition one or more of the ingredients selected from thrombin, epinephrine, collagen, calcium salts, and pH adjusting agents. Also useful are materials to promote degranulation or preserve platelets, additional growth factors or growth factor inhibitors, small molecule pharmaceuticals such as NSAIDS, steroids, and anti-infective agents. The invention also relates to the method wherein the platelet composition comprises platelets obtained from the patient.

In an aspect, the invention relates to the method wherein the patient's tissue where cancer may occur includes, but not limited to, connective tissues, prostate, lung, colon, bladder, skin, pancreas, blood cells, breast, ovary, gonad, brain or any combination thereof. In a preferred embodiment, a tissue in a patient where cancer may occur and is subject to methods and compositions as described herein is brain.

In an aspect, the invention relates to the method wherein obtaining the platelet composition comprises: drawing blood from a human; and centrifuging the blood to obtain a plasma-rich fraction. In an aspect, the invention relates to the method wherein the platelet composition comprises platelet-rich plasma. In an aspect, the invention relates to the method with the proviso that the platelet composition is substantially free from exogenous activators prior to its introduction into and around the region of the cancer in the patient. In an aspect, the invention relates to the method wherein the platelet composition comprises platelets obtained from the patient.

In yet another aspect, the invention relates to a composition comprising: platelet releasate wherein the composition is at a pH greater than or equal to physiological pH, and wherein the composition comprises substantially no unactivated platelets.

In a further aspect, the invention relates to a method of treating a cancer in brain comprising: obtaining a platelet composition; determining a location of the cancer; and introducing the platelet composition into and around the cancer. In some cases, substantially no activator is added to the platelet composition prior to its introduction into and around the cancer. In some cases, platelets are treated with energy waves, such as ultrasound, or with an activator to release platelet contents. In an aspect, the invention relates to the method wherein the platelet composition is minimally invasively introduced into and around the cancer. In an aspect, the invention relates to the method wherein the platelet composition comprises platelet-rich plasma. In an aspect, the invention relates to the method further comprising: mixing into the platelet composition substantially simultaneously with its minimally invasive introduction into and around the lesion one or more of the ingredients selected from thrombin, epinephrine, collagen, calcium salts, pH adjusting agents. Also useful are materials to promote degranulation or preserve platelets, additional growth factors or growth factor inhibitors, small molecule pharmaceuticals such as NSAIDS, steroids, and anti-infective agents. In an aspect, the invention relates to the method wherein obtaining the platelet composition comprises: drawing blood from a human; and centrifuging the blood to obtain a plasma-rich fraction. In an aspect, the invention relates to the method wherein the platelet composition is at or above physiological pH. In an aspect, the invention relates to the method wherein the platelet composition comprises platelets obtained from the patient.

In a further aspect, the invention relates to a method of treating a cancer occurring in or around connective tissues, prostate, lung, colon, bladder, skin, pancreas, blood cells, breast, ovary, gonad, brain or any combination thereof. Such method may comprise: obtaining a platelet composition; determining location, size and/or types of the cancer in need of treatment; and minimally invasively introducing the platelet composition into and around the cancer. In an aspect, the invention relates to the method wherein the platelet composition optionally includes platelet releasate. In an aspect, the invention relates to the method further comprising: mixing into the platelet composition one or more of the ingredients selected from thrombin, epinephrine, collagen, calcium salts, and pH adjusting agents. Also useful are materials to promote degranulation or preserve platelets, additional growth factors or growth factor inhibitors, small molecule pharmaceuticals such as NSAIDS, steroids, and anti-infective agents. In an aspect, the invention relates to the method wherein obtaining the platelet composition comprises: drawing blood from a human; and centrifuging the blood to obtain a plasma-rich fraction. In an aspect, the invention relates to the method wherein the platelet composition is at or above physiological pH. In an aspect, the invention relates to the method wherein the platelet composition comprises platelets obtained from the patient. In an aspect, the invention relates to the method with the proviso that the platelet composition is substantially free from exogenous activators prior to its introduction into and around the region of the cancer in the patient.

In one aspect of the invention the patient is treated with a combination of the PRP and conventional therapies such as surgery, radiation therapy, chemotherapy, immunological therapy and others. More specifically, PRP is prepared with blood that is obtained from the patient suffering from cancer or may be obtained from a healthy individual which has been tested against the patient being treated in order to determine that a range of matches occur with respect to the patient's serological typing. In one example, an appropriate formulation comprising PRP can be prepared for a patient who is subjected to a conventional surgery technique in order to surgically remove the cancerous tumor. After removal of at least part of the tumor the area where the tumor was removed from is treated with the formulation comprising PRP of the invention. In a case where chemotherapy is applied with the PRP formulation, the formulation can be applied simultaneously with the chemotherapy agent or it can be applied before or after the chemotherapy. In a case where radiation therapy is applied with the formulation, the formulation can be applied before, during or after the radiation is applied to the patient. This application of the formulation is beneficial in a number of different ways. The formulation can aid in improving wound healing. Further, the formulation can aid in modulating the inflammatory response. Lastly, the formulation can aid in modulating the growth of any cancer cells not removed via conventional means.

Thereafter, the patient may be repeatedly treated with the PRP formulation of the invention by periodically administering the formulation to the patient and, for example, specifically administering the formulation directly to the area for which the tumor was removed. The formulation may be a formulation comprising PRP. Alternatively, the formulation may be supplemented with one or more pharmaceutically active components such as recombinantly produced growth factors or cytokines. In addition, the formulation may contain other small molecules such as anti-inflammatory agents, antibiotics, anesthetics and the like.

It will be apparent to those skilled in the art that various modifications and variations can be made to the present invention without departing from the spirit or scope of the invention. Thus, it is intended that the present invention cover the modifications and variations of the present invention provided that they come within the scope of the appended claims and their equivalents. The following examples are illustrative of the present invention, and are not intended to limit it.

### EXAMPLES

### Example 1

PRP was prepared using a centrifuge unit made by Harvest (Plymouth, Mass.). (Similar units are available as The Biomet GPS system, the Depuy Symphony machine and the Medtronic Magellan machine.) Approximately 55 cc of blood was drawn from the patient using a standard sterile syringe, combined with 5 cc of a citrate dextrose solution for anticoagulation, and then spun down to isolate the platelets according to the manufacturer's protocol. These platelets were then resuspended in approximately 3 cc of plasma. The resulting platelet rich plasma solution (PRP) was quite acidic and was neutralized with using approximately 0.05 cc of an 8.4% sodium bicarbonate buffer per cc of PRP under sterile conditions to approximately physiologic pH of 7.4. The PRP was not activated through addition of exogenous activators. This PRP composition is referred to herein as autologous platelet extract (APEX).

### Example 2

Fifty cc of whole blood is drawn from a patient, and then prepared according to the method of Knighton, U.S. Pat. No. 5,165,938, column 3. The PRP is activated according to Knighton using recombinant human thrombin. The degranulated platelets are spun down and the releasate containing supernatant is recovered. The releasate may be optionally pH adjusted to a pH of 7.4 using sodium bicarbonate buffer.

Example 3

Thirty ml of whole blood were drawn from a patient. A platelet composition was prepared according to Example 1 of U.S. Pat. No. 5,510,102 to Cochrum, incorporated herein by reference in its entirety, except that no alginate is added to the platelet composition.

### Example 4

### Brain Cancer

A patient presents cancer in or near brain. The World Health Organization (WHO) has at least nine categories of primary brain tumors, which are based on the types of cells in which the tumors originate. Nine categories of primary brain tumors include (1) gliomas originating from glial cells, (2) infiltrative astrocytoma from astrocytes, (3) pilocytic astrocytoma from astrocytes, (4) oligodendroglioma from oligodendrocytes, (5) mixed oligoastrocytoma from oligodendocytes and astrocytes, (6) glioblastoma multiforme (GBM) from astrocytes and other brain cell types (astroblasts, spongioblasts), (7) ependymoma from ependymocytes, (8) medulloblastoma from primitive neural cell, and (9) meningioma from meninges. The secondary (metastatic) brain tumor is originated from cancer in other tissue or organ such as a liver, breast and colon.

The location, size, and kind of cancer in or near brain can be identified via various detection methods. Commonly used such detection methods include, but are not limited to, MRI, X-ray, CT scan, Positron Emission tomography (PET), Single Photon Emission Computed Tomography (SPECT), Electrical Impedance Tomography (EIT), Electrical Source Imaging (ESI), Magnetic Source Imaging (MSI), laser optical imaging, ultrasound techniques, biopsy, and immunohistochemical detection.

Using the technique of Example 1, an autologous platelet extract (APEX) is obtained. In some cases, APEX is treated with energy waves, such as ultrasound, to release platelet contents. In some cases, APEX is buffered to physiologic pH. In some cases, appropriate amount of sodium bicarbonate buffer (e.g. 8.4% sodium bicarbonate buffer) can be used to raise the pH to or slightly above 7.4. The extract may or may not need to be activated through the addition of exogenous agent(s).

The APEX is then introduced into or near the area of cancer via a catheter, needle or any functionally equivalent thereof. The APEX may be applied into the animal body alone or in combination with other components including physiological buffer, chemotherapy agents, growth factors, growth factor inhibitors, NSAIDS, steroids, anti-infectives, and some combination thereof. Such buffer may include, but not limited to, sodium bicarbonate, calcium gluconate, choline chloride, dextrose, EGTA, HEPES, maleic acid, MOPS, PIPES, sucrose, TES, TRIS BASE, TRIS-HCl, and urea. The APEX may also be combined with an implantable device such as a stent.

Via various imaging methods available in the art such as MRI guidance, the APEX is introduced into or near the area of cancer. In some embodiments, treatment of APEX can be done along with other treatment methods such as surgery, radiation and chemotherapy. For instance, APEX may reduce development of cancer, i.e. decreasing the size of cancer, reducing metastasis of cancer, reducing angiogenesis near the cancer and others. In some other instance, the APEX may assist recovery of non-cancerous cells when surgery, radiation and/or chemotherapy are applied and thereby some healthy, non-cancer cells are damaged.

The size, location and metastasis of the cancer can be regularly monitored via various methods as described above and the follow-up treatment of APEX can be further determined accordingly.

Alternatively, cancer cells can be obtained from the cancer and grown in an APEX medium. These in vitro-cultured cancer cells could be reintroduced back into the body to attack and kill the remaining tumor. Without intending to be limited by theory, it is hypothesized that the APEX media has either the ability to cause tumor cell apoptosis (cell death) in vivo or it may have the ability to transform cancer cells into normal cells.

Still alternatively, healthy, non-cancerous cells that are grown in the media containing APEX may be reintroduced to the area of cancer. For this, a researcher or clinician can obtain healthy, non-cancer cells from the patient or another healthy individual. In one example, such non-cancerous cells can be obtained in or near the area where the cancer resides. Using the technique of Example 1, an autologous platelet extract (APEX) is obtained and buffered to physiologic pH. The cells are grown in a media rich in the APEX in various conditions and dilutions. The APEX may be used to help grow and differentiate any tissue or cell line in vitro. When such in vitro-cultured cells are reintroduced to the area where normal cells are damaged due to surgery, radiation and/or chemotherapy, the reintroduced cells may help damaged cells to recover. Such recovery of normal cells may help treatment of cancer.

### Example 5

### Neoplastic Tissue

A patient presents with either a benign or malignant tumor or process. Using the technique of Example 1, an autologous platelet extract (APEX) is obtained and buffered to physiologic pH. The APEX can be used either in vivo or in vitro to initiate or induce tumor cell death.

Specifically, the APEX is injected into a solid tumor with CT or MRI guidance via a small catheter. Alternatively, cancer cells after being grown in an APEX media could be reintroduced back into the body to attack and kill the remaining tumor. Without intending to be limited by theory, it is hypothesized that the APEX media has either the ability to cause tumor cell apoptosis (cell death) in vivo or it may have the ability to transform cancer cells into normal cells.

### Example 6

### Cell Cultures of Non-Cancerous Cells

A researcher or clinician wishes to grow healthy, non-cancer cells in an in vitro culture system. Using the technique of Example 1, an autologous platelet extract (APEX) is obtained and buffered to physiologic pH.

The cells are then isolated and grown in a media rich in the APEX in various conditions and dilutions. The APEX may be used to help grow and differentiate any tissue or cell line in vivo and in vitro. When such in vitro-cultured cells are reintroduced to the area where normal cells are damaged due to surgery, radiation and/or chemotherapy, the reintroduced cells may help damaged cells to recover. Such recovery of normal cells may help treatment of cancer in any tissues.

### Example 7

### Cell Cultures of Cancerous Cells

A researcher or clinician wishes to grow cancerous cells in an in vitro culture system. Using the technique of Example 1, the APEX is obtained and buffered to physiologic pH.

The cells are obtained from the cancer in a patient and then become grown in a media rich in the APEX in various conditions and dilutions. These in vitro-cultured cancer cells could be reintroduced back into the body to attack and kill the remaining tumor. Without intending to be limited by theory, it is hypothesized that the APEX media has either the ability to cause tumor cell apoptosis (cell death) in vivo or it may have the ability to transform cancer cells into normal cells.

### Example 8

### Treatment of Brain Cancer in Human Glioblastoma Cells with APEX

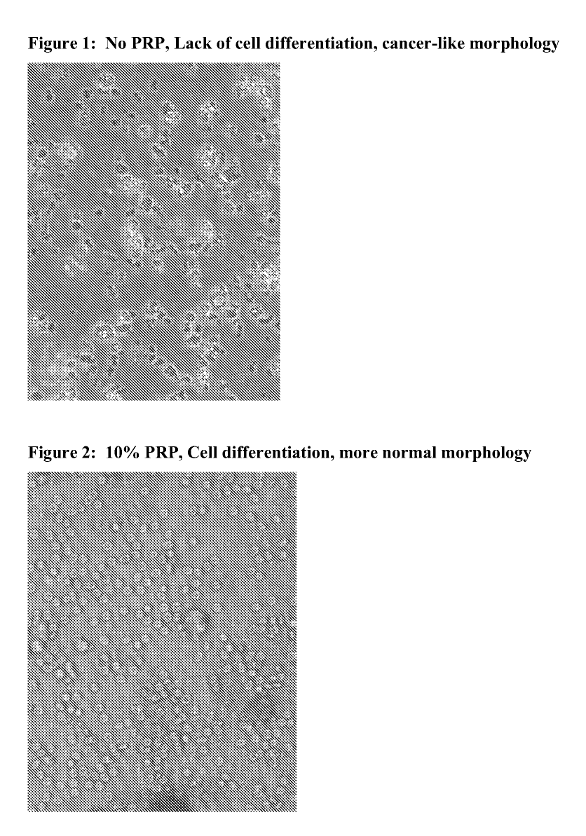

The cancer cells chosen for the experiment were glioblastoma cells which is an aggressive brain cancer. Patients with this form of brain cancer have a median survival time of 3 months if no treatment is provided. As can be seen in FIG. 1, human glioblastoma cells have an undifferentiated, cancer-like morphology.

PRP was prepared from whole blood and then exposed to energy waves to break open the platelets and obtain the platelet releasate. The energy waves were ultrasonic and were applied for 8 seconds. Human glioblastoma cancer cells were grown in cell culture in control media and 10% buffered using platelet rich plasma (PRP) for 10 days. The PRP was buffered using sodium bicarbonate to physiologic pH, around 7.4.

No exogenous activator was used. Specifically, no thrombin, calcium or epinephrine was added to the PRP or cultures.

At 10 days after incubation with PRP, photomicrographs were taken to analyze the morphologic characteristics of the cancer cells. At that time, marked differences in the structure of the cells were clear under microscopic examination. Differentiation into more normal cells, possibly astrocytes, was observed. See FIGS. 1-3.

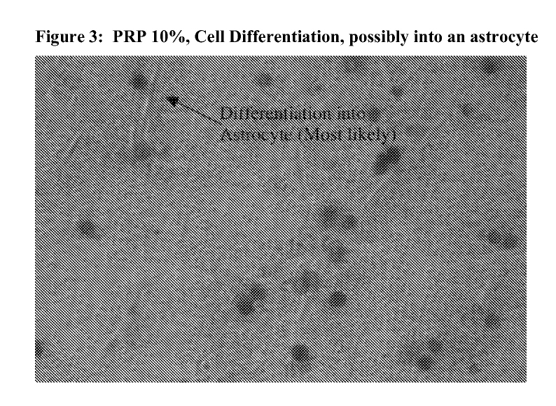

After 10 days culture in the presence of 10% platelet releasate, the morphology had normalized (FIG. 2). A possible astrocyte is shown in FIG. 3. The platelet releasate-treated cancer cells appear to actually revert to a normal morphology as indicated by FIGS. 2 and 3.

### Example 9

### Breast Cancer

In order to treat cancer present in or near breast of a patient, an APEX composition is prepared as described in Example 1. In some cases, appropriate amount of sodium bicarbonate buffer (e.g. 8.4% sodium bicarbonate buffer) can be used to raise the pH to or slightly above 7.4. The extract is not activated through the addition of exogenous agent(s).

The APEX is then introduced into the area of cancer via a catheter, needle or any functionally equivalent thereof. The APEX may also be combined with an implantable device such as a stent.

### Example 10

### Prostate Cancer

A patient presents cancer in or near a prostate. This cancer could be of any stage. Using the technique of Example 1, an autologous platelet extract (APEX) is obtained and buffered to physiologic pH.

The area of cancer in or near the prostate can be identified by various available diagnostic methods such as imaging methods. Once the cancer is identified, then the APEX is introduced into the area of cancer or near the cancer via a catheter, needle or any functionally equivalent thereof. This can be done a single time or it may require multiple injections. The size of the cancer can be regularly monitored and the follow-up treatment of APEX can be further determined.

### Example 11

### Cancer in Connective Tissues

In general, sarcomas are cancers that arise from the cells that hold the body together. These could be cells related to muscles, nerves, bones, fat, tendons, cartilage, or other forms of “connective tissues”. There are hundreds of different kinds of sarcomas, which come from different kinds of cells.

For example, a patient presents osteosarcoma, which is cancer in bone. Using the technique of Example 1, an autologous platelet extract (APEX) is obtained and buffered to physiologic pH.

The area of cancer can be identified by various diagnostic methods including X-ray guidance and then the APEX is introduced into or near tissues having cancer via a catheter, needle or any functionally equivalent thereof. This procedure could be done alone or in combination with a thermal/radiofrequency ablation procedure. The APEX could also be injected into a vertebral body that has sustained a compression fracture with or without the use of a balloon prior to injection.

### Example 12

### Cancer in Any Internal Organ

A patient presents cancerous cell or tissue growth in or near pancreas and/or any internal organ. Such internal organ may include, but not limited to, a pancreas, small intestine, large intestine, stomach, lung, liver, esophagus and others. Using the technique of Example 1, an autologous platelet extract (APEX) is obtained and buffered to physiologic pH.

Via various imaging methods available in the art such as CT guidance and conscious sedation, the APEX is introduced via a small gauge catheter into the target organ having cancer, e.g. pancreas.

### Example 13

### Cancer in Spinal Cord

A patient presents cancer in or near spinal cord. Using the technique of Example 1, an autologous platelet extract (APEX) is obtained and buffered to physiologic pH.

Via various imaging methods available in the art such as MRI guidance and conscious sedation, the APEX is introduced into or near the area of cancer. The APEX may reduce development of cancer as well as assist recovery of non-cancerous cells when surgery or radiation is applied with the APEX.

It will be understood by those of skill in the art that numerous and various modifications can be made without departing from the spirit of the present invention. Therefore, it should be clearly understood that the forms of the present invention are illustrative only and are not intended to limit the scope of the present invention.

1. A method for treating a cancer of an individual comprising:
identifying an area of cancerous tissue to be treated;
withdrawing whole blood from the individual;
preparing platelet rich plasma from the whole blood; and
delivering the platelet rich plasma to the area of cancerous tissue thereby treating cancer.

2. The method of claim 1, wherein the platelet rich plasma is delivered into and around an area where the cancer is located.

3. The method of claim 1, wherein delivering the platelet rich plasma comprises delivering the platelet rich plasma using a catheter, needle or any functional equivalent thereof.

4. The method of claim 1, wherein delivering the platelet rich plasma comprises delivering three to five cc of the platelet rich plasma.

5. The method of claim 1, wherein the prepared platelet rich plasma is treated with energy waves prior to delivery.

6. The method of claim 1, further comprising buffering the platelet rich plasma to a physiological pH.

7. The method of claim 6, wherein the physiological pH is between about 7.3 and 7.5.

8. The method of claim 6, wherein buffering the platelet rich plasma comprises buffering with a buffer selected from the group consisting of sodium bicarbonate, calcium gluconate, choline chloride, dextrose, EGTA, HEPES, maleic acid, MOPS, PIPES, sucrose, TES, TRIS BASE, TRIS-HCl, and urea.

9. The method of claim 1, wherein delivering the platelet rich plasma comprises injecting the platelet rich plasma.

10. The method of claim 1, further comprising adding an agent to the platelet rich plasma, wherein the agent is selected from the group consisting of growth factors, growth factor inhibitors, NSAIDS, steroids, anti-infectives, and some combination thereof.

11. The method of claim 1, wherein the platelet rich plasma is depleted in neutrophils.

12. A method for treating a cancer of an individual comprising:
obtaining cancer cells from the individual;
culturing the cancer cells in a medium comprising platelet extract; and
introducing at least some of the cancer cells cultured in the medium comprising platelet extract to the area of cancerous tissue in the individual thereby treating cancer.

13. The method of claim 12, wherein the platelet extract is an autologous platelet extract obtained from the individual.

14. The method of claim 12, wherein the platelet extract is buffered to a physiological pH.

15. The method of claim 14, wherein the physiological pH is between about 7.3 and 7.5.

16. The method of claim 14, wherein buffering the platelet extract comprises buffering with a buffer selected from the group consisting of sodium bicarbonate, calcium gluconate, choline chloride, dextrose, EGTA, HEPES, maleic acid, MOPS, PIPES, sucrose, TES, TRIS BASE, TRIS-HCl, and urea.

17. The method of claim 12, further comprising adding an agent to the platelet extract, wherein the agent is selected from the group consisting of growth factors, growth factor inhibitors, NSAIDS, steroids, anti-infectives, and some combination thereof.

18. The method of claim 12, wherein the platelet extract is depleted in neutrophils.

19. The method of claim 12, wherein introducing the cancer cells comprises introducing the cells using a catheter, needle or any functional equivalent thereof.

20. A method for treating a cancer comprising:
obtaining non-cancerous cells from a first individual;
culturing the non-cancerous cells in a medium comprising platelet extract; and
introducing at least some of the non-cancerous cells cultured in the medium comprising platelet extract to the area of cancerous tissue in the first or a second individual thereby treating cancer.

21. The method of claim 20, wherein the platelet extract is an autologous platelet extract obtained from the first individual.

22. The method of claim 20, wherein the platelet extract is buffered to a physiological pH.

23. The method of claim 22, wherein the physiological pH is between about 7.3 and 7.5.

24. The method of claim 22, wherein buffering the platelet extract comprises buffering with a buffer selected from the group consisting of sodium bicarbonate, calcium gluconate, choline chloride, dextrose, EGTA, HEPES, maleic acid, MOPS, PIPES, sucrose, TES, TRIS BASE, TRIS-HCl, and urea.

25. The method of claim 20, further comprising adding an agent to the platelet extract, wherein the agent is selected from the group consisting of growth factors, growth factor inhibitors, NSAIDS, steroids, anti-infectives, and some combination thereof.

26. The method of claim 20, wherein the platelet extract is depleted in neutrophils.

27. The method of claim 20, wherein introducing the non-cancerous cells comprises introducing the cells using a catheter, needle or any functional equivalent thereof.

28-34. (canceled)
